(19) United States
(12) Patent Application Publication
(10) Pub. No.: US 2023/0414067 A1
(43) Pub. Date: Dec. 28, 2023

(54) IMAGE DISPLAY DEVICE, METHOD FOR OPERATING IMAGE DISPLAY DEVICE, AND ENDOSCOPE SYSTEM

(71) Applicant: OLYMPUS MEDICAL SYSTEMS CORP., Tokyo (JP)

(72) Inventor: Goki NUMATA, Tokyo (JP)

(73) Assignee: OLYMPUS MEDICAL SYSTEMS CORP., Tokyo (JP)

(21) Appl. No.: 18/367,736

(22) Filed: Sep. 13, 2023

Related U.S. Application Data
(63) Continuation of application No. PCT/JP2021/011257, filed on Mar. 18, 2021.

(51) Int. Cl. A61B 1/00; H02J 7/00

(52) U.S. Cl. CPC A61B 1/00036; H02J 7/0048; A61B 1/00009; A61B 1/00048; A61B 1/00055; A61B 1/00006

(57) ABSTRACT

An image display device includes: a plurality of circuits configured to process image signals received from an endoscope, and display an endoscope image on a display; a battery configured to supply electric power to the plurality of circuits; and a processor. The processor switches a control mode in which the electric power is supplied to the plurality of circuits, from a normal mode to any one of a plurality of standby modes in which power consumption is set to be lower than in the normal mode; and the processor switches the control mode, in a case where date and time are out of a preset scheduled use period, to a second standby mode in which the power consumption is set to be lower than in a first standby mode of a case where the date and time are within the scheduled use period.

### CROSS REFERENCE TO RELATED APPLICATION

This application is a continuation application of PCT/IP2021/011.257 filed on Mar. 18, 2021, the entire contents of which are incorporated herein by this reference.

### BACKGROUND OF THE INVENTION

### 1. Field of the Invention

An embodiment of the present invention relates to an image display device that receives image signals from an endoscope and displays an endoscope image on a display, a method for operating the image display device, and an endoscope system including an endoscope and the image display device.

### 2. Description of the Related Art

An endoscope system for observing an inside of a body cavity or the like of a patient is widely used. An endoscope image is transmitted from an endoscope via a cable to a processor that performs signal processing.

In Japanese Patent Application Laid-Open Publication No. 2016459021, a wireless endoscope has been developed that is equipped with a rechargeable battery and transmits an endoscope image as a wireless signal to an image display device which is a receiver. In an endoscope system including the wireless endoscope, the wireless signal transmitted by the endoscope is received by a receiver that is paired (establishment of communication). The wireless endoscope which does not have a cable is excellent in operability.

In International Publication No. 2018/207537, a wireless endoscope is disclosed that achieves both a short return time and low power consumption, by switching a control mode between a normal mode and a standby mode with low power consumption, in which pairing is maintained.

In recent years, a portable monitor has been developed, which is a battery-driven type receiver that receives an image signal from a wireless endoscope, performs image processing, and displays an image on a display. An endoscope system including the portable monitor is excellent in portability and does not require power supply from an external power source; and accordingly, a place of use is not limited.

### SUMMARY OF THE INVENTION

An image display device according to an embodiment of the present invention includes: a plurality of circuits configured to process image signals received from an endoscope, and display an endoscope image on a display; a battery configured to supply electric power to the plurality of circuits; and a processor. The processor switches a control mode in which the electric power is supplied to the plurality of circuits, from a normal mode of a case where the image display device receives the image signals, to any one of a plurality of standby modes in which power consumption is set to be lower than in the normal mode; and the processor switches the control mode, in a case where date and time are out of a preset scheduled use period, to a second standby mode in which the power consumption is set to be lower than in a first standby mode of a case where the date and time are within the scheduled use period.

A method for operating an image display device according to an embodiment of the present invention includes: when switching a control mode in which a battery supplies electric power to a plurality of circuits that process image signals received from an endoscope and display an endoscope image, from a normal mode in a case of receiving the image signals, to any one of a plurality of standby modes in which power consumption is set to be lower than in the normal mode, switching the control mode, in a case where date and time are out of a preset scheduled use period, to a second standby mode in which the power consumption is set to be lower than in a first standby mode of a case where the date and time are within the scheduled use period.

An endoscope system according to an embodiment of the present invention includes: an endoscope; and an image display device. The image display device includes: a communication unit configured to receive image signals from the endoscope, a plurality of circuits that include an image processing unit configured to process the image signals, and a display configured to display an endoscope image which the image processing unit outputs; a battery configured to supply electric power to the plurality of circuits; and a processor configured to switch a control mode in which the electric power is supplied to the plurality of circuits, from a normal mode of a case where the image display device receives the image signals, to any one of a plurality of standby modes in which power consumption is set to be lower than in the normal mode. The processor switches the control mode, in a case where date and time are out of a preset scheduled use period, to a second standby mode in which power consumption is set to be lower than in a first standby mode of a case where the date and time are within the scheduled use period.

### BRIEF DESCRIPTION OF THE DRAWINGS

FIG. 1 is a perspective view of an endoscope system including a receiver according to an embodiment;

FIG. 2 is a configuration diagram of the receiver according to the embodiment;

FIG. 3 is one example of a use schedule of the endoscope system;

FIG. 4 is a flow chart of a method for operating the receiver according to the embodiment,

FIG. 5 is a configuration diagram of a receiver according to first and second modifications of the embodiment;

FIG. 6 is a flow chart of an endoscope system including the receiver according to the first modification of the embodiment;

FIG. 7 is a configuration diagram of a receiver according to a third modification of the embodiment; and

FIG. 8 is a flow chart of an endoscope system including a receiver according to a fourth modification of the embodiment.

### DETAILED DESCRIPTION OF THE PREFERRED EMBODIMENT

### Embodiment

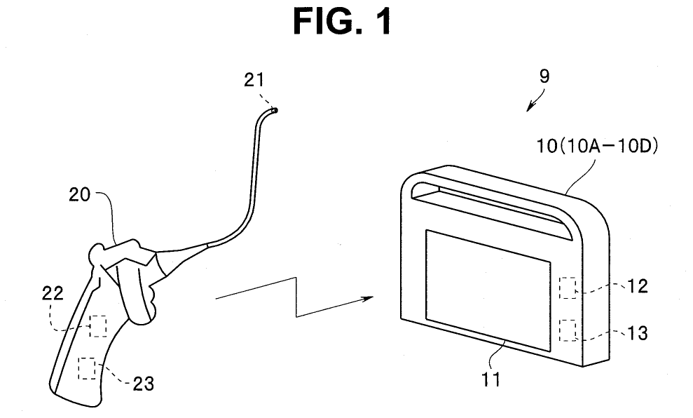

An image display device 10 (receiver 10) according to an embodiment shown in FIG. 1 constitutes an endoscope system 9 together with an endoscope 20.

The drawings of the embodiment and the like are schematic views. A relationship between a thickness and a width of each portion, a ratio among the thicknesses of respective portions and the like are different from a relationship and a ratio of actual portions. Also, among the mutual drawings, portions are contained in which relationships and ratios between mutual dimensions are different. Illustration and affixation of reference characters are omitted in some components.

The endoscope 20 is a wireless endoscope that is driven by a battely 22. The endoscope 20 includes, for example, an image pickup section 21 at a distal end portion of an elongated insertion portion. The endoscope 20 may be a flexible endoscope having a flexible insertion portion or a rigid endoscope having a rigid insertion portion. The endoscope 20 may be used for medical purposes or industrial purposes.

The receiver 10 is an image display device driven by the battery 12. The receiver 10 is a portable monitor having a display 11 configured to display an endoscope image.

A configuration related to wireless communication of the endoscope system 9 will be described below. The wireless communication is performed between the endoscope 20 and the receiver 10 which uses, for example, a 5 GFIz band or a 60 GHz band.

The endoscope image which the image pickup section 21 of the endoscope 20 has acquired is wirelessly transmitted by a communication section 23 as an image signal. A communication section 13 of the receiver 10 receives the image signal and displays the endoscope image on the display 11.

### =Configuration of Receiver>

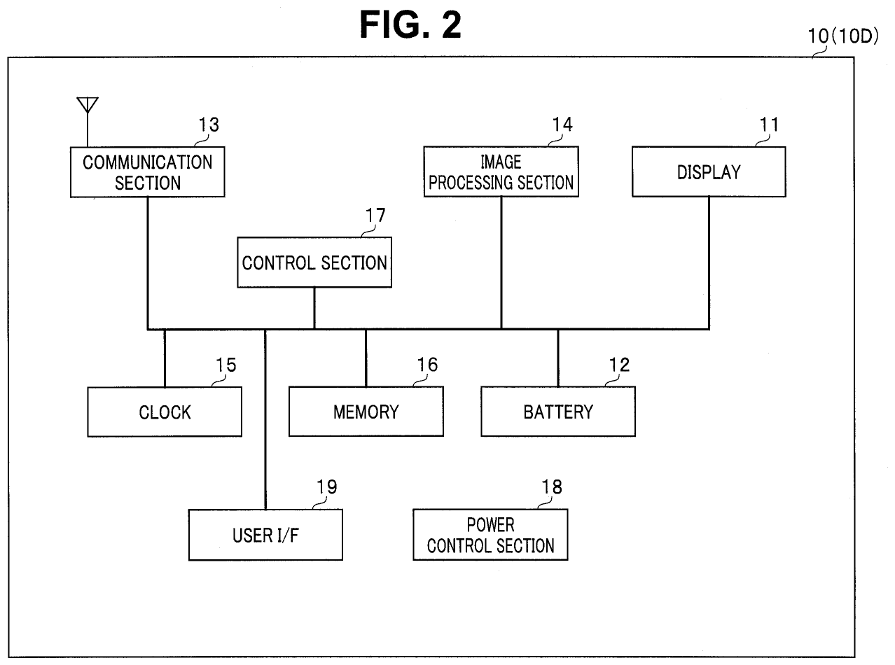

A configuration of the receiver 10 will be described with reference to FIG. 2.

The receiver 10 includes the display 11, the battery 12, the communication section (communication unit) 13, an image processing section (image processing unit) 14, a clock 15, a memory 16, a control section 17 (processor 17), a power control section (power control unit) 18, and a user interface (user 1/F) 19. In other words, the receiver 10 includes a plurality of circuits that consume electric power, in the display 11, the communication section 13, the image processing section 14, the user interface 19 and the like.

The battery 12 generates electric power necessary for driving the receiver 10. The battery 12 is, for example, a lithium-ion secondary battery. The receiver 10 is driven by the battery 12 similarly to the endoscope 20, and also is light in weight, and accordingly, can be easily carded.

The communication section 13 wirelessly receives the image signal from the endoscope 20. The image processing section 14 performs predetermined processing on the image signal which the communication section 13 has received, and outputs the endoscope image.

The display 11 displays the endoscope image which the image processing section 14 has outputted. The user interface 19 is an unillustrated keyboard, a switch or the like, and supplies an operation signal based on a user operation to the control section 17. The display 11 may be a touch panel type of user interface 19.

The control section 17 controls the whole receiver. The power control section 18 controls the electric power which the battery 12 supplies. The clock 15 has functions of not only a time point but also a calendar (date and day of the week). The memory 16 is formed of a semiconductor, and stores data of a use schedule and the like, which will be described later.

The control section 17 may have a function of the power control section 18. At least any one of the image processing section 14, the control section 17, and the power control section 18 may be configured by a CPU that operates by software, or may be configured by a dedicated hardware circuit. The clock 15 and the memory 16 may be functions of the CPU. A program that causes a computer (CPU) to execute the receiver 10 may be stored in a non-transitory computer-readable storage medium.

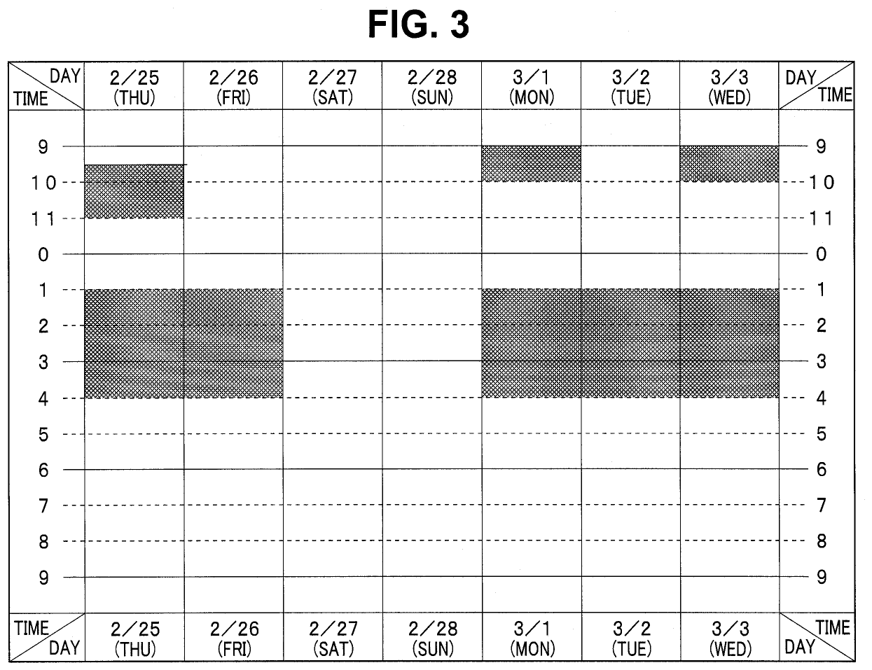

FIG. 3 is an example of the use schedule of the endoscope system 9 (receiver 10 and endoscope 20). The date and time in which a time frame is displayed in black in the drawing indicate that the date and time are within the scheduled use period. For example, on February 25 (Tuesday), the time frames of 9:30 AM to 11:00 AM and 01:00 PM to 04:00 PM are use schedules. The use schedule may be a daily schedule, a monthly schedule, or even a monthly or longer schedule.

For example, the use schedule is inputted by a user in advance with the use of the user interface 19, and is stored in the memory 16. When a plurality of users access a host computer from respective terminals, select the date and time on which the use of the endoscope system 9 is not scheduled, and input data to the host computer, thus reserved data may be transmitted to the receiver 10.

The power control section 18 switches a control mode between a normal mode and a plurality of standby modes in which power consumption is set to be lower than in the normal mode. Furthermore, the power control section 18 switches the control mode, in a case where the date and time of the clock 15 are out of the scheduled use period, to a second standby mode in which the power consumption is set to be lower than in a first standby mode of a case where the date and time are within the scheduled use period.

In the present embodiment, the power control section 18 controls a power supply with the use of three control modes of the normal mode, the first standby mode, and the second standby mode. In the normal mode when the communication section 13 receives the image signal, the power control section 18 supplies electric power sent from the battery 12 to all the circuits of the receiver 10. The first standby mode and the second standby mode are control modes of the case where the endoscope 20 is not used for observation of a subject (of the case where the communication section does not receive the image signal), in which the power consumption is reduced as compared to the normal mode, but pairing is continued.

For example, in the first standby mode, electric power is not supplied to the display 11. In the second standby mode, electric power is not supplied to the display 11 and the user interface 19. It takes a relatively long time for the endoscope 20 and the receiver 10 to execute pairing. However, the pairing is continued in the first standby mode or the second standby mode, and accordingly, it takes short time for the first standby mode or the second standby mode to return to the normal mode.

The receiver 10 is a battery-driven type of portable monitor, but electric power is supplied from the battery 12 according to the use schedule; and accordingly, both a short recovery time and low power consumption are achieved.

### <Method for Operating Receiver>

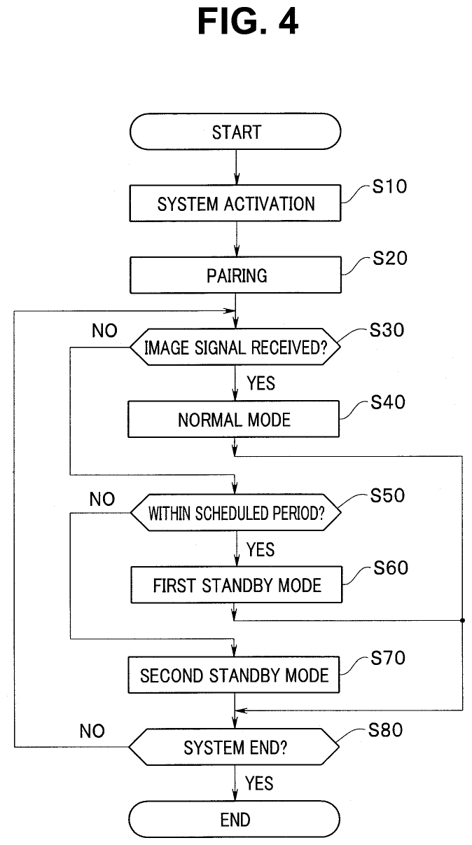

A method for operating the receiver 10 will be described with reference to a flow chart of FIG. 4.

### <Step S10>System Activation

When the endoscope system 9 is activated by an operation of a user, electric power is supplied from the battery 12 of the receiver 10 to each electric member (circuit section). Note that the electric power is supplied from the battery 12 or an unillustrated auxiliary battery to some of the plurality of circuit sections, for example, the clock 15, even when the endoscope system 9 is in a stopped state. Before activation, the battery 12 is connected to, for example, a commercial power supply by wire, and is charged.

### <Step S20>Fairing

For example, the communication section 13 executes pairing for establishing mutual communication with the endoscope 20 according to an initial setting which is stored in the memory 16. There are many wireless devices in an operating room. The communication section 13 searches for a vacant channel so as to be capable of avoiding interference and securing a necessary band, changes a setting as needed, and establishes the communication. For this reason, there is the case where the pairing takes a long time.

Note that in a case where the pairing is not completed even after a predetermined time has elapsed, it is preferable for the control section 17 to generate, for example, a warning sound and terminate the system.

### <Step S30>Normal Image Signal Received?

When the communication section 13 which has been paired with the communication section 23 of the endoscope 20 has received a normal image signal from the endoscope 20 (YES), the power control section 18 controls the batter 12 in the normal mode, in step S40. When the endoscope 20 is not used, the power control section 18 automatically stops the power supply to an illumination circuit, the image pickup section 21, or the like in order to alleviate the power consumption. Then, the image signal which the communication section 13 receives becomes, for example, an abnormal black image signal in which the whole of the image is black (NO). A size of the black image signal is extremely small as compared to a size of the normal image signal. Note that the power control section 18 may determine whether or not the image signal is normal with the use of an AI technique.

### <Step S40>Normal Mode

In the normal mode, the state continues in which electric power is supplied to each circuit from the battery 12. The endoscope image which the image processing section 14 outputs is displayed on the display 11.

### <Step S50>within Scheduled Period?

When the normal image has not been received, the power control section 18 compares the date and time indicated by the clock 15, with the use schedule of the endoscope system 9 (receiver 10). Note that in a case where the communication section 13 is wirelessly connected to the host computer in which the use schedule is stored, for example, the host computer may perform comparison processing, and only a result may be transmitted to the receiver 10.

In a case where the date and time are within the scheduled use period (YES), the power control section 18 controls the receiver 10 with the first standby mode, in step S60. On the other hand, in a case where the date and time are out of the scheduled use period (NO), the power control section 18 controls the receiver 10 with the second standby mode, in step S70.

### <Step S60>First Standby Mode

In the first standby mode, for example, the power supply to the display 11 from the battery 12 is stopped. 6<Step S70>Second standby mode

In the second standby mode, for example, the power supply to the display 11 and the user interface 19 from the battery 12 is stopped. In the second standby mode in a period out of the scheduled use period, the power consumption is set to be lower than in the first standby mode at the scheduled use period. In the first standby mode and the second standby mode, the pairing is maintained, and accordingly the return time to the normal mode is short.

### <Step S80>End?

The processing from step S30 is repeated until the operation of the endoscope system 9 is completed by the operation of the user.

As described above, the method for operating the image display device according to the present embodiment includes:
when switching the control mode in which the battery supplies the electric power to the plurality of circuits that process the image signals received from the endoscope and display the endoscope image, from the normal mode in the case where the image display device has received the image signals, to any one of the plurality of standby modes in which the power consumption is set to be lower than in the normal mode, switching the control mode, in the case where the date and time are out of a preset scheduled use period, to the second standby mode in which the power consumption is set to be lower than in the first standby mode of the case where the date and time are within the scheduled use period.

### Modifications of Embodiment

Receivers (image display devices) 10A to 10D of modifications of the embodiment are similar to the receiver 10 of the embodiment, and components having the same functions are denoted by the same reference characters and description thereof will be omitted.

### <First Modification>

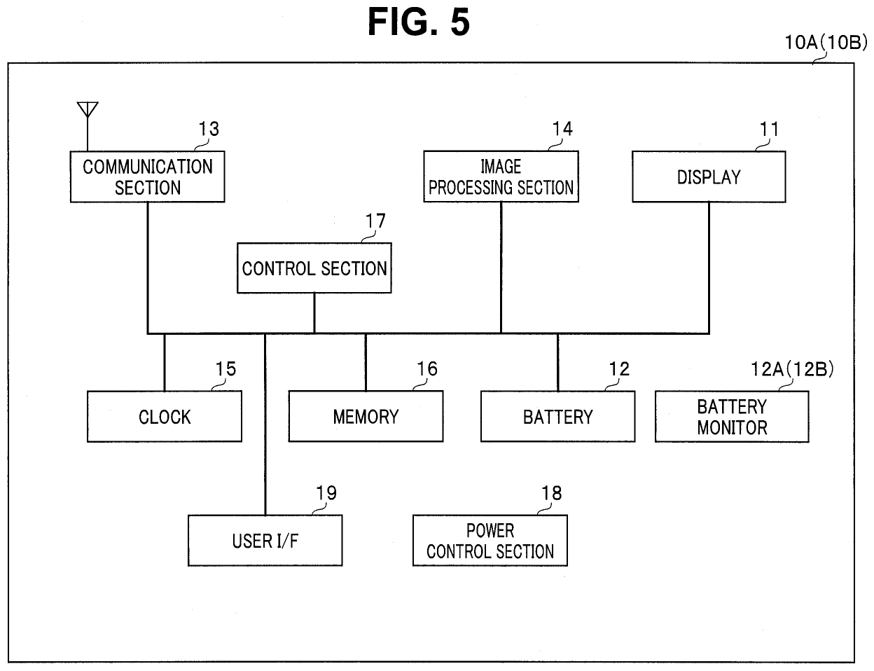

The receiver 10A of the present modification shown in FIG. 5 includes a battery monitor 12A that acquires a remaining capacity of the battery 12. In the receiver 10A, the power control section 18 controls the power supply in any one of four control modes including the normal mode and three standby modes.

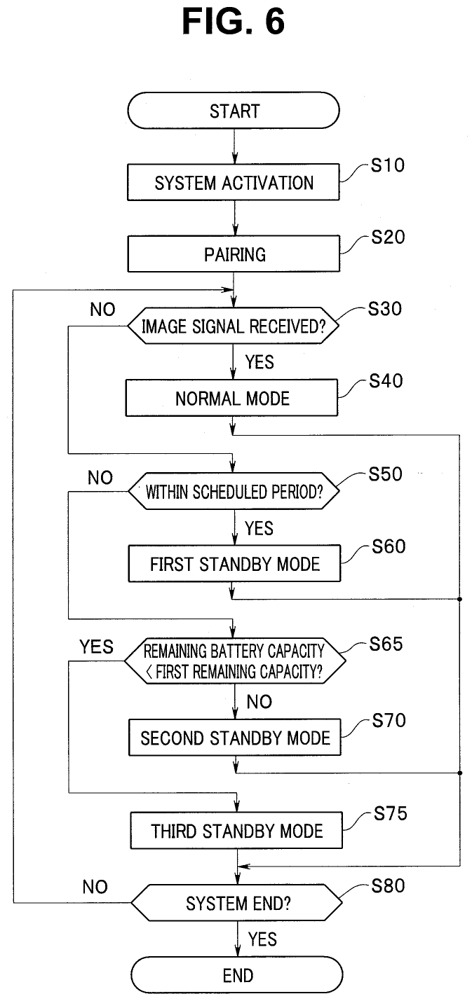

A method for operating the receiver 10A will be described with reference to a flow chart of FIG. 6.

### <Steps S10 to S60>

Steps S10 to S60 of the receiver 10A are the same as the steps S10 to S60 in the flow chart (FIG. 4) of the receiver 10.

### <Step S65>

In a case where the remaining capacity of the battery 12 which the battery monitor 12A has acquired is less than a first remaining capacity (YES), the power control section 18 switches the control mode from the normal mode to a third standby mode, in step S75. In a case where the remaining capacity of the battery 12 is the first remaining capacity or more (NO), the power control section 18 switches the control mode from the normal mode to the second standby mode, in step S70. The first remaining capacity is, for example, 30% of a full charge capacity.

### <Step S70>Second Standby Mode

Steps S10 to S70 of the receiver 10A are the same as the steps S10 to S70 in the flow chart (FIG. 4) of the receiver 10. In the second standby mode, the power consumption is set to be lower than in the first standby mode.

### <Step S75>Third Standby Mode

In the third standby mode, for example, the power supply to the display 11, the user interface 19, and the image processing section 14 from the battery 12 is stopped. In the third standby mode, the power consumption is set to be lower than in the second standby mode.

In the receiver 10A, the battery 12 is controlled according to the remaining capacity of the battery 12, in the third standby mode in which the power consumption is set to be lower than in the second standby mode, and accordingly, the power consumption is lower than in the receiver 10; and the use of the receiver can also be safely stopped.

Note that in a case where the remaining capacity of the battery 12 is less than a second remaining capacity, it is preferable for the power control section 18 to generate an alarm. The second remaining capacity is a battery capacity necessary for safely completing the use of the endoscope system 9 with a margin. The second remaining capacity is, for example, 10% of the full charge capacity.

### <Second Modification>

The receiver 10B of the present modification shown in FIG. 5 further includes a battery monitor 12B that acquires a used time after the battery 12 has been fully charged.

A method for operating the receiver 10B differs from the method for operating the receiver 10A shown in FIG. 6, only in processing in step S65. In the receiver 10B, it is determined in step S65 whether or not a “discharge time after the battery 12 has been fully charged exceeds a predetermined first amount of time”.

The power control section 18 switches the control mode, in a case where the discharge time exceeds the first amount of time, to the third standby mode in which the power consumption is set to be lower than in the second standby mode. A content of each standby mode is the same as in the receiver 10A. The first amount of time is, for example, 30% of the time after the battery 12 has been fully charged and before the receiver 10B cannot be used. The first amount of time is inputted in advance by the user, for example, with the use of the user interface 19, and is stored in the memory 16.

In the receiver 10B, the battery 12 is controlled according to the discharge time of the battery 12, in the third standby mode in which the power consumption is set to be lower than in the second standby mode, and accordingly, the power consumption is lower than in the receiver 10; and the use of the receiver 10B can also be safely stopped. The pairing is maintained in any standby modes, and accordingly, the return time of the receiver 10B to the normal mode is short.

### <Third Modification>

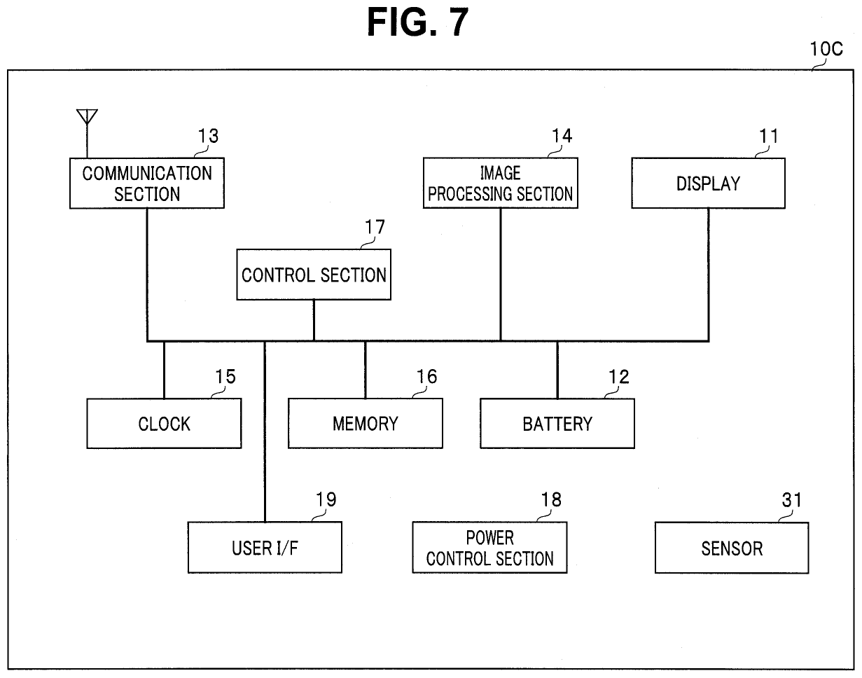

As shown in FIG. 7, the receiver 10C of the present modification further includes a sensor 31 for detecting movement of the receiver 10C. The sensor 31 is, for example, an acceleration sensor or a GPS sensor, and when the receiver 10C is not stationary, the power control section 18 determines that the receiver 10C is moving and the endoscope system 9 is not in use.

The power control section 18 switches the control mode to the third standby mode in which the power consumption is set to be lower than in the second standby mode, while the receiver 10C is moving, on the basis of a detection result of the sensor 31 for detecting the movement.

In the receiver 10C, the battery 12 is controlled in the third standby mode in which the power consumption is set to be lower than in the second standby mode, while the receiver 10C is moving, and accordingly, the power consumption is lower than in the receiver 10.

### <Fourth Modification>

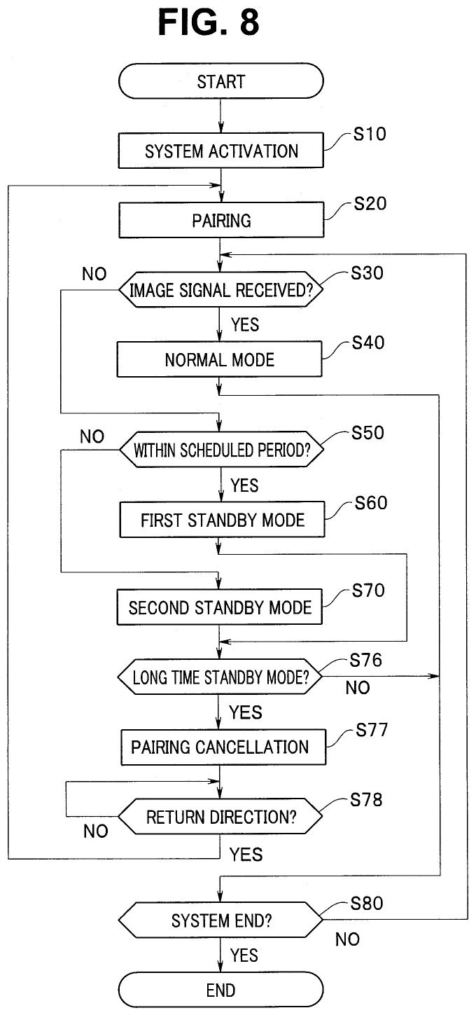

A method for operating the receiver 10D of the present modification will be described with reference to a flow chart of FIG. 8. In the receiver 10D, in a case where the control mode is the first or second standby mode for a long time, the power control section 18 cancels the pairing with the endoscope 20.

### <Steps S10 to S70>

Steps S10 to S70 of the receiver 10D are the same as the steps S10 to S70 in the flow chart (FIG. 4) of the receiver 10.

### <Steps S76 to S77>

The power control section 18 of the receiver 10D cancels the pairing with the endoscope 20 in step S77, in a case where the control mode is the first or second standby mode for a period longer than a predetermined second amount of time (YES). In this case, the power control section 18 may further stop the power supply to the communication section 13 from the battery 12. The second amount of time is inputted in advance by the user, for example, with the use of the user interface 19, and is stored in the memory 16.

### <Step S78>

The power control section 18 continues the pairing cancellation until the return is directed (NO).

In a case where the control mode is any of the standby modes for a long time, the battery 12 does not consume a useless electric power, and accordingly, the power consumption of the receiver 10D is lower than in the receiver 10.

Note that in a case where the control mode is any of the standby modes in a period longer than a predetermined third amount of time, the power control section 18 may interrupt the power supply to each electric member (circuit) from the battery 12, and stop the operation of the receiver 10D, The third amount of time is inputted in advance by the user, for example, with the use of the user interface 19, and is stored in the memory 16.

In a plurality of standby modes in a wireless device of the present invention, the electric members (circuits) to which the power supply is stopped are not limited to the display 11, the user interface 19, and the image processing section 14, but can be selected from a plurality of electric members (circuits/components) that consume the electric power and are built in the receiver 10.

The receiver of the present invention may have configurations of a plurality of modifications. For example, the receiver of the present invention may have all the configurations of modifications 1 to 4.

The present invention is not limited to the above described embodiment and the like, and various changes, modifications and the like can be made without changing the gist of the present invention.

What is claimed is:

1. An image display device comprising:
a plurality of circuits configured to process image signals received from an endoscope, and display an endoscope image on a display;
a battery configured to supply electric power to the plurality of circuits; and
a processor, wherein
the processor switches a control mode in which the electric power is supplied to the plurality of circuits, from a normal mode of a case where the image display device receives the image signals, to any one of a plurality of standby modes in which power consumption is set to be lower than in the normal mode; and
the processor switches the control mode, in a case where date and time are out of a preset scheduled use period, to a second standby mode in which the power consumption is set to be lower than in a first standby mode of a case where the date and time are within the scheduled use period.

2. The image display device according to claim 1, further comprising:
a battery monitor configured to acquire a remaining capacity of the battery, wherein
the plurality of standby modes include a third standby mode in which the power consumption is set to be lower than in the second standby mode, and
the processor switches the control mode to the third standby mode, in a case where the remaining capacity is less than a first remaining capacity.

3. The image display device according to claim 2, wherein
the processor generates an alarm in a case where the remaining capacity is less than a second remaining capacity.

4. The image display device according to claim 2, wherein
the processor switches the control mode to the third standby mode, in a case where a discharge time after the battery is fully charged exceeds a first amount of time.

5. The image display device according to claim 1, further comprising:
a sensor configured to detect movement, wherein
the plurality of standby modes include a third standby mode in which the power consumption is set to be lower than in the second standby mode, and
the processor switches the control mode to the third standby mode during the movement.

6. The image display device according to claim 1, wherein
the processor cancels pairing with the endoscope, in a case of being paired with the endoscope and in a case where the control mode is any one of the plurality of standby modes for a period longer than a second amount of time.

7. The image display device according to claim 6, wherein
the processor interrupts a power supply from the battery, in a case where the control mode is any one of the plurality of standby modes for a period longer than a third amount of time.

8. A method for operating an image display device, comprising:
when switching a control mode in which a battery supplies electric power to a plurality of circuits that process image signals received from an endoscope and display an endoscope image, from a normal mode in a case of receiving the image signals, to any one of a plurality of standby modes in which power consumption is set to be lower than in the normal mode,
switching the control mode, in a case where date and time are out of a preset scheduled use period, to a second standby mode in which the power consumption is set to be lower than in a first standby mode of a case where the date and time are within the scheduled use period.

9. The method for operating an image display device according to claim 8, further comprising:
switching the control mode to a third standby mode in which the power consumption is set to be lower than in the second standby mode, in a case where remaining capacity of the battery is less than a first remaining capacity.

10. The method for operating an image display device according to claim 8, further comprising:
generating an alarm, in a case where the remaining capacity of the battery is less than a second remaining capacity.

11. The method for operating an image display device according to claim 8, further comprising:
switching the control mode to a third standby mode in which the power consumption is set to be lower than in the second standby mode, in a case where a discharge time of the battery after the battery is fully charged exceeds a first amount of time.

12. The method for operating an image display device according to claim 8, further comprising:
switching the control mode to a third standby mode in which the power consumption is set to be lower than in the second standby mode, in a case of movement, according to a detection result of a sensor which detects the movement.

13. The method for operating an image display device according to claim 8, further comprising:
cancelling pairing with the endoscope, in a case where the control mode is any one of the plurality of standby modes for a period longer than a second amount of time.

14. The method for operating an image display device according to claim 8, further comprising:
interrupting a power supply, in a case where the control mode is any one of the plurality of standby modes for a period longer than a third amount of time.

15. An endoscope system comprising:
an endoscope; and
an image display device, wherein
the image display device comprises:
a communication unit configured to receive image signals from the endoscope;
a plurality of circuits that include an image processing unit configured to process the image signals, and a display configured to display an endoscope image which the image processing unit outputs;
a battery configured to supply electric power to the plurality of circuits; and
a processor configured to switch a control mode in which the electric power is supplied to the plurality of circuits, from a normal mode of a case where the image display device receives the image signals, to any one of a plurality of standby modes in which power consumption is set to be lower than in the normal mode, wherein
the processor switches the control mode, in a case where date and time are out of a preset scheduled use period, to a second standby mode in which power consumption is se to be lower than in a first standby mode of a case where the date and time are within the scheduled use period.

16. The endoscope system according to claim 15, wherein
the image display device further comprises a battery monitor configured to acquire a remaining capacity of the battery, and
the processor switches the control mode to a third standby mode in which power consumption is se to be lower than in the second standby mode, in a case where the remaining capacity of the battery is less than a first remaining capacity.

17. The endoscope system according to claim 15, wherein
the processor generates an alarm in a case where the remaining capacity of the battery is less than a second remaining capacity.

18. The endoscope system according to claim 15, wherein
the image display device further comprises a battery monitor configured to acquire a discharge time after the battery is fully charged, and
the processor switches the control mode to a third standby mode in which the power consumption is set to be lower than in the second standby mode, in a case where the discharge time exceeds a first amount of time.

19. The endoscope system according to claim 15, wherein
the image display device further comprises a sensor configured to detect movement, and
the processor switches the control mode to a third standby mode in which the power consumption is set to be lower than in the second standby mode, during the movement.

20. The endoscope system according to claim 15, wherein
the processor interrupts a power supply, in a case where the control mode is any one of the plurality of standby modes for a period longer than a third amount of time.
